(19) United States
(12) Patent Application Publication
(10) Pub. No.: US 2004/0213856 A1
(43) Pub. Date: Oct. 28, 2004

(54) Compositions and methods for treatment of multiple myeloma

(76) Inventor: Eric A Scheinbart, Savannah, GA (US)

Correspondence Address: LICATLA & TYRRELL P.C., 66 E. MAIN STREET, MARLTON, NJ, 08053, US

(21) Appl. No.: 10/258,663

(22) PCT Filed: Apr. 26, 2001
(86) PCT No.: PCT/US01/40602

Related U.S. Application Data
(60) Provisional application No. 60/200,681, filed on Apr. 29, 2000.
(60) Provisional application No. 60/232,721, filed on Sep. 15, 2000.

(51) Int. Cl. A61K 35/55; A61K 35/78; A61K 35/84; A61K 31/715

(52) U.S. Cl. 424/568; 424/195.15; 514/54; 424/725

(57) ABSTRACT

A composition and method for treatment of multiple myeloma and drug-resistant pneumonia are provided. The composition is a combination of natural products that stimulate immune system function and restore well-being in patients.

### BACKGROUND OF THE INVENTION

Multiple myeloma is a neoplastic disease characterized by infiltration of bone and bone marrow by myeloma cells forming multiple tumor masses that lead to pathological fractures. The condition is usually progressive and fatal. Symptoms include anemia, renal damage and high globulin levels in blood and increased susceptibility to bacterial infections. The impaired abnormal immunoglobulin production observed in multiple myeloma may be due to the presence of a monocyte or macrophage that suppresses the maturation of normal B-lymphocytes into antibody secreting cells. Life expectancy is related to extent of the disease at diagnosis and response to treatment. The median life expectancy of responding patients is two years. High levels of myeloma (M) protein in serum or urine, bone lesions, hypercalcemia, pancytopenia, and renal failure are unfavorable signs. The standard treatments for the disease are the highly toxic chemotherapy agents. Although there is no cure, these drugs can prolong and improvefthe quality of life. However, the side effects seen with these agents often limit their use.

Glucans, including beta-1,3-D-glucan, are known to have a variety of beneficial effects on human physiology. They are known to stimulate macrophage activity, and as a result stimulate immune system function. Glucans have been used to treat a wide variety of diseases including heart disease, AIDS, and infectious diseases as a general group. Studies have shown that glucans provide a protective effect against an extensive list of microorganisms including Staphylococcus aureus, Escherichia coli, Candida albicans, Pneumocystis carinii, Listeria monocytogenes, Leishmania donovani, Herpes simplex, and Ascaris sp. (see review in Drug and Therapy Perspectives 1996. 8:6-7). Numerous studies support the idea that an antibiotic and a macrophage activator, such as glucan, work synergistically.

Glucan has also been reported to have a protective effect on bone marrow during radiation therapy, with glucan shown to enhance hematopoietic recovery by regenerating the host's ability to resist life-threatening opportunistic infections (Research Report: Radioprotective Effect of Oral Administration of Beta-1,3-D-glucan. Bethesda, Md.: Armed Forces Radiobiology Research Institute, 1987). It has been demonstrated that host resistance to opportunistic infection in glucan-treated irradiated animals is enhanced even prior to the detection of significant hematopoietic regeneration. This early enhanced resistance to microbial invasion was correlated with enhanced and/or prolonged macrophage, but not granulocyte, function.

It has now been found that beta-1,3-D-glucan in combination with several other herbal products is an effective treatment for multiple myeloma, as well as vancomycin-resistant S. aureus pneumonia.

### SUMMARY OF THE INVENTION

An object of the present invention is a composition for the treatment of multiple myeloma and vancomycin-resistant pneumonia which comprises beta-1,3-D-glucan, a Maitake mushroom extract, a Maharishi Amrit Kalash-4 herbal product, a Maharishi Amrit Kalash-5 herbal product, a thymus extract, and a general nutritional supplement.

Another object of the present invention is a method for treating multiple myeloma and vancomycin-resistant pneumonia in a patient comprising administering to said patient an effective amount of the composition which comprises beta-1,3-D-glucan, a Maitake mushroom extract, a Maharishi Amrit Kalash-4 herbal product, a Maharishi Amrit Kalash-5 herbal product, a thymus extract, and a general nutritional supplement.

### DETAILED DESCRIPTION OF THE INVENTION

The present invention is a composition and method for treating multiple myeloma and vancomycin-resistant pneumonia in patients with active disease. The composition comprises a mixture of natural products. Natural products are currently used by patients with a variety of diseases as either a complement to or in place of conventional pharmaceutical treatments. Natural products often have the advantage of being biologically active without producing the high levels of toxicity often associated with standard treatments for diseases such as multiple myeloma. The premise of the development of the composition of the present invention was that a properly functioning immune system is essential for optimal health. Further, the macrophage, which is a cell that plays an essential role in initiating and maintaining the immune response, can be targeted by compounds such as the glucans, including beta-1,3-D-glucan.

The composition of the present invention is a combination therapy which involves administration of six different natural products. These products are beta-1,3-D-glucan, a Maitake mushroom extract, a Maharishi Amrit Kalash-4 herbal product, a Maharishi Amrit Kalash-5 herbal product, a thymus extract, and a general nutritional supplement.

Beta-1,3-D-glucan, like the other naturally-occurring glucans, is a known immunomodulator that can be isolated from the cell walls of a common yeast called S. cerevisiae. Studies have shown that beta-1,3-D-glucan has the ability to stimulated macrophage function, which leads to a cascade effect on cells of the immune system and results in an overall stimulation of the immune response. Therefore, since the goal of development of the composition of the present invention was to stimulate immune system function, the use of this glucan provided a compound that has this role.

In addition to beta-1,3-D-glucan, Maitake mushroom extract was included in the composition of the present invention to maximize macrophage stimulation. Maitake mushrooms (Grifola frondosa) have been used in Chinese Traditional medicine for thousands of years. Extracts of this mushroom have been reported to have anti-tumor activity, anti-HIV activity, and anti-viral activity. The D-fraction of this extract, which has beta-glucan as a major component, appears to possess a high level of anti-tumor activity and was therefore included in the composition of the present invention. Unlike other medicinal mushrooms, the beta-glucan from the Maitake mushrooms has a unique structure; a 1,6 main chain having greater degree of 1,3 branches. It has been suggested that the greater degree of branching leads to a greater likelihood for immune system activation. Like beta-1,3-D-glucan, Maitake mushroom extract has been reported to inhibit growth of existing cancer cells and when taken in conjunction with other chemotherapeutics to enhance activity of those agents (Nanba, H. 1997. Ann. NY Acad. Sci. 768:243-245). Therefore, the combination of immuno-modulating activity of Maitake mushroom extract and beta-glucan was exploited in the composition of the present invention.

The composition of the present invention also contains two compounds known as free radical scavengers, Maharishi Amrit Kalash-4 (MAK-4) and Maharishi Amrit Kalash-5 (MAK-5). These are complex herbal mixtures that are rich in vitamin C, vitamin E, beta-carotenes, polyphenols, bioflavonoids, and riboflavin. Research has shown that MAK-4 and MAK-5 are 1000 times more potent at scavenging free radicals than vitamins C and E alone and are the only substances known to protect against the entire spectrum of free radicals (Sharma, H. M. et al. 1992. Pharmacol. Biochem. Behav. 43:1175-1182). Like beta-glucan and Maitake mushroom extract, MAK-4 and MAK-5 have been shown to reduce the side effects associated with chemotherapy (Misra, N. C. et al. 1994. In: Proceedings of the XVI International Cancer Congress, Rao, R. S. and D,L Sanghvi (eds), pp. 3099-3102), and to be potent anti-tumor agents (Dileepan, K. N. et al. 1993. Biochem. Arch. 9:365-374). Therefore, these two herbal mixtures were included in the composition of the present invention based on their potential anti-cancer effects.

The thymus is the major gland of the immune system and is responsible for regulation of many immune factors. Function and size of this gland is known to decline with age and is adversely affected by stress and severe or prolonged illness. Typically, thymic hormone levels are very low in those who are prone to cancer and chronic diseases. T-lymphocytes, which derive their name from their dependence on the thymus, are part of the immune system's front line of defense against cancer as well as infection. Studies have shown the usefulness of thymic extracts in restoring immunological deficits that are consistently encountered in cancer patients (Whitaker, J. 1997. Whitaker's Health and Healing 7:1-2). The thymus produces numerous hormone-like peptides that act as chemical messengers activating T-cells and Natural Killer cells to seek and destroy cancer cells (Lapcevic, J. 1997. Townsend Letter for Doctors and Patients, February/March, 70-72). Therefore, a thymic extract was included in the composition of the present invention.

The last component of the composition of the present invention was a nutritional supplement known as Gero-Vital GH-3. This supplement was used as a way to aid in the enhancement of the body and spirit of the patient being treated.

A 56 year old white male was diagnosed in September 1998 with multiple myeloma. The patient underwent treatment for the disease that included three unsuccessful bone marrow transplants that followed three total body radiations. During this time of treatment, the patient's red blood cell and platelet counts, as well as renal function, continued to decline, despite repeated transfusions and treatment with compounds known to affect red cell and platelet counts, Procrit and Neupogen. In December 1999 the patient was admitted to the hospital for pneumonia and sepsis where sputum and blood cultures were positive for vancomycin-resistant S. aureus pneumonia. The patient was instructed to return home and prepare to die as his disease was terminal and near the end of its course. The patient was bedridden, not eating and having difficulty swallowing.

After returning home the patient was started on an alternative therapy regimen that consisted of the following:

1. Beta-1,3-D-glucan (500 mg by mouth three times a day)

2. Maitake D-Fraction mushroom extract (2 droppers full three times a day)

3. MAK-4 herbal concentrate (one teaspoon three times a day)

4. MAK-5 herbal tablet (one tablet three times a day)

5. A thymic extract product, Pro-Boost Thymic formula (one packet given sublingually three times a day)

6. A general nutritional supplement, Gero-Vital GH-3 (one tablet three times a day)

This regimen was given each day and was accompanied by institution of minimum nutrient targets in the diet by inclusion of daily portions of fresh fruits and vegetables, as well as supplementation with a liquid protein supplement as tolerated.

Within one week, ulcerations of the oral mucosa had resolved and the patient was feeling better and was ambulatory. Two months after initiating the therapy with the composition of the present invention, the patient was re-evaluated by an oncologist and was diagnosed as in remission for his disease and free of pneumonia.

The patient progressively improved over the next few months and at one point decided on his own to discontinue the therapy with the composition of the present invention, an action that resulted in a decline in his health and laboratory indices of disease. The patient then re-initiated the therapy and his health and laboratory indices of disease improved. Now two years after initiation of the therapy with the composition of the present invention the patient's electrolytes, CBC and differential, immunoglobulins, electrophoresis and bone marrow aspirate were all within the normal range. He also had a negative MRI and CT scan for disease. The patient's lifestyle had returned to near normal.

Therefore, the composition of the present invention, an alternative therapy that has combined immunostimulants, anti-oxidants, thymic protein and nutritional supplement is an effective treatment for multiple myeloma as well as vancomycin-resistant pneumonia in humans. The present invention is also a method for treating multiple myeloma and vancomycin-resistant pneumonia in a patient comprising administering to said patient an effective amount of the composition which comprises beta-1,3-D-glucan, a Maitake mushroom extract, a Maharishi Amrit Kalash-4 herbal product, a Maharishi Amrit Kalash-5 herbal product, a thymus extract, and a general nutritional supplement. In the context of the present invention an “effective amount” is an amount shown to have activity to cause remission of these diseases in humans as described above. The present invention has the advantages of being effective while without significant toxicity and also of being inexpensive compared to standard treatments for these diseases.

What is claimed is:

1. A composition for the treatment of multiple myeloma and vancomycin-resistant pneumonia comprising beta-1,3-D-glucan, a Maitake mushroom extract, a Maharishi Amrit Kalash-4 herbal product, a Maharishi Amrit Kalash-5 herbal product, a thymus extract, and a general nutritional supplement.

2. A method for treating multiple myeloma and vancomycin-resistant pneumonia in a patient comprising administering to said patient an effective amount of the composition which comprises beta-1,3-D-glucan, a Maitake mushroom extract, a Maharishi Amrit Kalash-4 herbal product, a Maharishi Amrit Ralash-5 herbal product, a thymus extract, and a general nutritional supplement.
